(19) United States
(12) Patent Application Publication
(10) Pub. No.: US 2007/0238953 A1
(43) Pub. Date: Oct. 11, 2007

(54) Combined ultrasonic imaging and spectroscopic molecular analysis

(75) Inventors: Gerald LUCASSEN, Eindhoven (NL); Nico WILLARD, Valkenswaard (NL); Sieglinde NEERKEN, Eindhoven (NL); Ties VAN BOMMEL, Horst (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/570,444

(22) PCT Filed: Jun. 13, 2005
(86) PCT No.: PCT/IB05/51943

§ 371 Date: Dec. 12, 2006

(30) Foreign Application Priority Data
Jun. 17, 2004 (EP) 04102767.3

(51) Int. Cl. A61B 5/05; A61B 8/00

(52) U.S. Cl. 600/407; 600/437

(57) ABSTRACT

The invention provides an apparatus for combined imaging and analyzing of a biological structure by means of ultrasonic and spectroscopic techniques. The inventive diagnostic apparatus is adapted for simultaneous acquisition of ultrasonic echo signals and surface enhanced Raman spectroscopic signals. The invention makes particular use of a contrast agent that on the one hand provides enhanced reflection efficiency for ultrasonic echo signals and on the other hand enables surface enhanced Raman spectroscopy. The contrast agent comprises conventional microbubbles as well as solid metal nano-particles or combinations thereof. The invention therefore effectively provides non-invasive in vivo analysis of blood as well as detection and visualization of a blood flow.

The present invention relates to the field of imaging and analyzing of a biological structure by means of ultrasonic and spectroscopic techniques.

Within the framework of medical diagnostics analysis of blood is of great importance. Analysis of blood refers to information of the blood flow within a cardiovascular system as well as to an analysis of the composition of blood of a patient. In order to detect and visualize the flow of blood, nowadays ultrasonography as well as ultrasound imaging is successfully applied. These ultrasonic imaging techniques make effectively use of ultrasound contrast agents (UCAs) that are injected into the blood stream of a patient. Typically, UCAs are based on gas filled microbubbles featuring remarkable reflection properties regarding to ultrasound. Therefore, by injection of a contrast agent into a cardiovascular system of a patient and subsequently making use of an ultrasonic imaging system effectively allows to detect and to visualize the blood flow of a patient. Blood flow information is of crucial importance in the cardiac critical care, in emergency situations as well as in surgery to check and to monitor a patient's cardiac function.

Generally, ultrasonic imaging systems provide sufficient information related to the location of blood vessels as well as information related to the flow properties of blood flowing through the blood vessels. However, by means of ultrasonic imaging techniques it is generally not possible to specify certain properties of blood, such as the composition of blood or a proportion of designated substances in the blood, e.g. cholesterol or glucose. Typically, the composition of blood is investigated in vitro. This means that a blood sample is taken from a patient and subsequently submitted to a chemical lab performing a composition analysis of the sample. This rather time intensive and sometimes cumbersome investigation procedure can be effectively circumvented by making use of non-invasive blood analysis that is based on a spectroscopic technique. An analysis apparatus for such a non-invasive blood analysis is for example disclosed in WO 02/057759.

The article “Intravascular ultrasound combined with Raman spectroscopy to localize and quantify cholesterol and calcium salts in atherosclerotic coronary arteries” by Römer et al. published in “Arteriosclerosis, Thrombosis, and Vascular Biology”, February 2000; 20:pages 478-483 discloses a combination of intravascular ultrasound (IVUS) and Raman spectroscopy to evaluate an intact arterial wall. Here, IVUS images were collected in vitro from human coronary arterial segments in various stages of disease. The images were divided into radial segments, each of which was classified visually as calcified or non calcified tissue. The arteries were opened longitudinally, and Raman spectra were collected from locations at 0.5 mm intervals across the arterial luminal circumference. The spectra were used to calculate the chemical composition of the arterial wall at the examined locations. Generally, locations containing large amounts of calcium salts, as determined with Raman spectroscopy, were classified as calcified with IVUS.

During IVUS measurements, calcifications were identified by the shadows behind echo-dense areas of the real-time by the US images. These arterial planes were marked with a curved surgical needle positioned opposite the calcification and further serves to ensure that the Raman spectroscopic measurements would be obtained from the same axial plane. The Raman spectroscopic measurements were performed off line on separate occasions to prohibit bias. This article focuses on assessing arterial wall architecture by making use of intravascular ultrasound and applying Raman spectroscopic techniques in order to quantify compounds in homogenized arterial tissue.

The present invention aims to provide an apparatus for locating, tracing and analyzing of a biological structure or substance within a cardiovascular system of a patient.

The present invention provides an apparatus comprising an ultrasonic imaging system for imaging of a volume of interest and a spectroscopic system for determining a property of a biological structure being located within the volume of interest. Hence the invention provides a combination of ultrasonic imaging technique and spectroscopic analysis in order to locate, trace and analyze a biological structure within e.g. a cardiovascular system of a patient. The apparatus is preferably designed for visualization of blood flow and simultaneous analysis of the composition of the blood. Moreover, the biological structure may also refer to a biological substance such as blood flowing through a blood vessel that is located within the volume of interest. Therefore, the inventive apparatus is designed for in vivo investigation of a volume of interest such as e.g. a blood vessel.

According to a further preferred embodiment of the invention, the apparatus comprises a flexible probe head that has at least an objective lens for directing an excitation beam into the volume of interest and for collecting return radiation from the volume of interest. The flexible probe head further has an ultrasonic detection element that serves as an ultrasonic imaging sensor for acquisition of ultrasonic echo signals from the volume of interest during a period of time. The probe head combines means for acquisition of spectroscopic data and means for acquisition of ultrasonic data that are indicative of molecular composition and location and/or flow information of a substance within the volume of interest, respectively.

The objective lens of the flexible probe head serves to collimate the excitation beam into the volume of interest. Preferably, the objective lens is part of a confocal optical arrangement for confocally focusing the excitation beam into the volume of interest. Making use of e.g. Raman spectroscopy, the excitation beam is typically in the near infrared (NIR) range. At least a portion of the deposited excitation radiation interacts with the substance that is located within the volume of interest and becomes subject to an inelastic scattering process, such as e.g. a Stokes or anti-Stokes scattering process.

Inelastically scattered radiation is typically frequency shifted and is therefore indicative of vibrational energy levels of the molecules of the substance. Inelastically scattered and hence frequency shifted radiation re-enters the probe head as return radiation from the volume of interest. Spectral analysis of the return radiation finally provides information of the molecular composition of the substance. Applying such a spectroscopic technique for blood analysis allows for example to determine the relative weights of cholesterol, glucose, blood oxygen and other analytes of the blood.

The ultrasonic detection element of the flexible probe head is adapted for acquisition of ultrasonic echo signals from the volume of interest during a period of time. In this way the volume of interest can be effectively visualized by means of ultrasonic imaging. Moreover, by monitoring the volume of interest during a period of time an entire sequence of ultrasonic images is obtained which allows for an effective observation of dynamic changes that may apply during the period of time. Hence, the ultrasonic imaging system is adapted to provide e.g. flow information of blood flowing through a blood vessel. Preferably, the ultrasonic detection element serves as a basis for an ultrasonic sensor and can be implemented as any kind of ultrasonic detection element that is adapted to receive ultrasonic echo signals from the volume of interest and to transform received signals into an electrical and/or optical signal for further processing. The ultrasonic sensor effectively provides ultrasonic imaging of the volume of interest and can therefore effectively be implemented as an ultrasonic imaging sensor.

According to a further preferred embodiment of the invention, the objective lens is integrated into the ultrasonic detection element of the probe head. Preferably, the ultrasonic sensor or ultrasonic detection element features a cut out for the objective lens. The ultrasonic sensor as well as the objective lens are an integrated part of the bottom side of the preferably hand held flexible probe head. When in operation the ultrasonic sensor is in contact with the skin of the patient and the objective lens is in close proximity to the surface of the skin of the patient. Additionally, the ultrasonic sensor may not only adapted to acquire ultrasonic echo signals but also to transmit ultrasonic signals to the volume of interest. The ultrasonic sensor may therefore be implemented as an ultrasonic transducer.

According to a further preferred embodiment of the invention, the apparatus further comprises a base station that has a spectroscopic analysis unit that is adapted to perform a spectroscopic analysis of the return radiation. The base station further has an ultrasonic analysis unit that is adapted to generate an image of the volume of interest on the basis of the acquired ultrasonic echo signals. Moreover, the base station is connected to the probe head of the apparatus in order to receive return radiation as well as ultrasonic echo signals that are acquired by the probe head. The base station provides effective means for universal signal analysis of either spectroscopic signals being indicative of the composition of the substance as well as ultrasonic signals being indicative of the location of the substance and e.g. flow information of blood. By implementing the spectroscopic as well as the ultrasonic analysis means of the apparatus into the base station, the flexible probe head can be designed in a compact way. Consequently, the probe head of the apparatus is preferably designed as a hand held device that is connected to the base station.

The connection between the flexible probe head and the base station typically provides optical transmission means, such as e.g. a single or a plurality of optical fibers providing bidirectional transmission of excitation and return radiation. Additionally, the connection has at least one electrical conducting element for transmission of electrical signals that are indicative of ultrasonic echo signals that are acquired by the ultrasonic sensor of the probe head.

According to a further preferred embodiment of the invention, the apparatus is adapted to perform spectroscopic analysis of the substance by means of the spectroscopic system and to simultaneously generate an image of the volume of interest by means of the ultrasonic imaging system. Here, simultaneous spectroscopic analysis and the generation of an ultrasonic image refers to at least partially overlapping time intervals for spectroscopic analysis and ultrasonic image generation. Alternatively, spectroscopic analysis of the substance and ultrasonic image generation may be performed sequentially. For example, an ultrasonic image or a sequence of ultrasonic images can be acquired and provided to a user of the apparatus. The user may then specify a particular region within the volume of interest to which a spectroscopic analysis has to be applied.

Moreover, the user may selectively initiate spectroscopic analysis of a designated region. No matter on whether spectroscopic analysis and ultrasonic image generation is performed simultaneously, partially simultaneously or even sequentially, the inventive apparatus is adapted to provide an ultrasonic image in combination with corresponding results of a spectroscopic analysis.

According to a further preferred embodiment of the invention, the flexible probe head of the apparatus is implemented as a hand held device that is adapted to be attached to the surface of the skin of the patient. This allows for non-invasive acquisition of ultrasonic echo signals as well as non-invasive acquisition of spectroscopic data. Such a non-invasive acquisition of diagnostic data is on the one hand comfortable for the patient and on the other hand minimizes a potential risk of inflicting an injury upon the patient during examination.

According to a further preferred embodiment of the invention, the flexible probe head is implemented as a catheter or an endoscope that are adapted to be inserted into the cardiovascular system of the patient. In this way an intravascular in vivo and in situ examination can be performed. This allows for example to localize intravascular plaque deposition in combination with blood analysis. Hence, a whole set of spectroscopic data acquired at various locations of a blood vessel can be effectively correlated to morphological information of the vascular wall of the blood vessel. Since the spectroscopic data is indicative of the molecular composition of the blood, a spatial variance of e.g. a particular analyte of the blood being correlated with e.g. atherosclerotic deposition at the vascular wall may be indicative of dangerous plaque rupture.

According to a further preferred embodiment of the invention, the ultrasonic imaging system is further adapted to acquire and to process ultrasonic echo signals from solid metal nano-particles being injected into the volume of interest as a contrast agent. Preferably, the solid metal nano-particles are bio-compatible, non toxic and have a diameter of between 1 nanometer and 100 nanometers, particularly between 1 nanometer and 50 nanometers. Especially when clustered, the metal nano-particles serve as acoustic reflectors due to their strong acoustic impedance difference with body tissue and have the advantage over commercial UCAs (microbubbles) of being stable and that they can be modified in the same way as current targeted contrast agents. Moreover, such a contrast agent can effectively be used with all forms of sonography, e.g. D-mode, Doppler shift sonography, etc.

Enhancement of the ultrasonic echo signal by means of a contrast agent and in particular by means of metal nano-particles as contrast agents can be obtained in a plurality of different configurations. The metal nano-particles are typically injected into the cardiovascular system of the patient. Enhancement of the ultrasonic echo signal is already obtained by the presence of the metal nano-particles in the blood stream of the patient. Moreover, signal enhancement arises when a plurality of nano-particles form a cluster. Additionally, when the metal nano-particles are targeted, i.e. they comprise a bio-target agent such as cell, tissue, microorganism, e.g. parasite, or bio-molecule, e.g. protein, DNA or RNA specific target agents, the metal nano-particles may adsorb to specific biological structures within the volume of interest. For example, the targeted nano-particles adsorb to a vascular wall. Additionally, the targeted nano-particles may adhere to commercial UCAs, e.g. microbubbles. Therefore, the metal nano-particles may either be used as a substitute of conventional ultrasonic contrast agents or in combination with ultrasonic contrast agents, such as microbubbles.

According to a further preferred embodiment of the invention, the spectroscopic system of the apparatus is further adapted to acquire and to process return radiation that is due to a surface enhanced Raman scattering process (SERS) induced by the solid metal nano-particles. Generally, a rather weak Raman signal being indicative of a molecules vibrational quantum states can be appreciably enhanced if the molecule is attached to a nanometer sized metal structure. Since the enhancement factor of surface enhanced Raman scattering (SERS) can be up to 14 orders of magnitude, SERS features an appreciably higher detection efficiency than conventional Raman spectroscopic techniques. For the present invention injection of solid metal nano-particles into the blood stream of a patient on the one hand provides a contrast agent for the ultrasonic imaging and on the other hand allows for surface enhanced Raman spectroscopy. Therefore, making use of metal nano-particles is advantageous for both spectroscopic analysis as well as for ultrasonic imaging.

In another aspect, the invention provides a probe head of an apparatus for imaging a volume of interest and for determining a property of a biological structure being located within the volume of interest. The inventive probe head comprises at least an objective lens for directing an excitation beam into the volume of interest and for collecting return radiation from the volume of interest. The probe head further comprises an ultrasonic sensor for acquisition of ultrasonic echo signals from the volume of interest during a period of time.

According to a further preferred embodiment of the invention, the objective lens is bordered by the ultrasonic sensor. Hence, the objective lens is designed as an integral part of the ultrasonic sensor. Both the ultrasonic sensor as well as the objective lens are typically arranged at the bottom side of the probe head facing the surface of the skin of a patient for acquisition of ultrasonic echo signals and spectroscopic radiation, respectively. For example, the ultrasonic sensor is implemented as a piezoelectric crystal that has a ring shaped geometry. The objective lens is then located in the center of this ring shaped piezoelectric crystal. In this way, the ultrasonic image acquisition volume as well as the spectroscopic signal acquisition volume may substantially overlap inherently, thus defining a combined data acquisition volume. Such an embodiment is advantageous because image and/or signal acquisition volumes for the ultrasonic and the spectroscopic systems have not to be combined manually.

In still another aspect, the invention provides a base station of an apparatus for imaging a volume of interest and for determining a property of a biological structure being located within the volume of interest. The base station comprises at least a spectroscopic analysis unit that is adapted to perform spectroscopic analysis of return radiation returning from the volume of interest. The base station further comprises an ultrasonic analysis unit that is adapted to generate an image of the volume of interest on the basis of ultrasonic echo signals from the volume of interest.

In still another aspect, the invention provides a method of imaging a volume of interest and for determining a property of a biological structure that is located within the volume of interest. The method comprises the steps of acquiring of ultrasonic echo signals from the volume of interest during a period of time, directing an excitation beam into the volume of interest and collecting return radiation from the volume of interest as well as performing spectroscopic analysis of the collected return radiation. Acquisition of ultrasonic echo signals from the volume of interest allows for generating an ultrasonic image of the volume of interest. By making use of e.g. a contrast agent injected into the cardiovascular system of a patient the flow of blood of the patient can be effectively visualized by means of the acquisition of ultrasonic echo signals. By collecting return radiation from the volume of interest and spectrally analyzing the collected return radiation a property of the biological structure that is located within the volume of interest can be effectively determined. Since the collected return radiation has been subject to inelastic scattering processes likes Stokes or anti-Stokes processes, its spectral analysis allows to determine the molecular composition of the biological structure.

According to a further preferred embodiment of the invention, the method further comprises injecting a contrast agent of solid metal nano-particles into the volume of interest. The contrast agent is adapted to enhance the ultrasonic echo signals and to simultaneously enhance the intensity of the return radiation. Therefore, the metal nano-particles serve as ultrasonic contrast agents as well as an effective means to stimulate surface enhanced Raman spectroscopy (SERS). Typically, the metal particles comprise a metal, which has an acoustic impedance above 35.105 g/cm²s, particularly above 50.105 g/cm²s, or a mixture of one or more metals. More particularly, according to the present invention, the metal particles are a noble metal, e.g. gold, silver, platinum. Optionally, the metal particles comprise a metal oxide or have a stable thin oxide layer or may have a bio-neutral biocompatible coating.

According to a further preferred embodiment of the invention, the metal nano-particle comprising a bio-targeted agent attached to the surface of the metal nano-particle. Bio-target agents are for example cell, tissue, microorganism, e.g. parasite or bio-molecule, e.g. protein, DNA or RNA specific target agents of which antibodies or fragments thereof are only one example. By means of bio-targeted agents the nano-particles may adsorb to the blood vessel wall or tissue. On the one hand by ultrasonic imaging of targeted nano-particles a blood vessel wall or specific tissue can be localized and the specific tissue or the blood vessel wall can be spectrally analyzed by SERS. Moreover, also blood flowing near the specific tissue or blood vessel wall can become subject to spectroscopic analysis.

Additionally, targeted nano-particles may accumulate to form a cluster of a plurality of nano-particles. An increase of the size of the contrast agents as given by such a cluster of nano-particles is particularly advantageous for reflection enhancement of ultrasonic echo-signals.

According to a further preferred embodiment of the invention, micro sized gas bubbles that are conventionally used as contrast agent for ultrasonic imaging may serve as a host for targeted metal nano particles. Hence, targeted nano particles are adapted to adhere to a membrane of these gas bubbles thus forming a complex that universally supports ultrasonic contrast enhancement as well as surface enhancement of spectroscopic signals. The gas bubbles and nano-particles therefore selectively enhance the efficiency of ultrasonic and spectroscopic data acquisition, respectively.

In the following preferred embodiments of the invention will be described by making reference to the drawings in which:

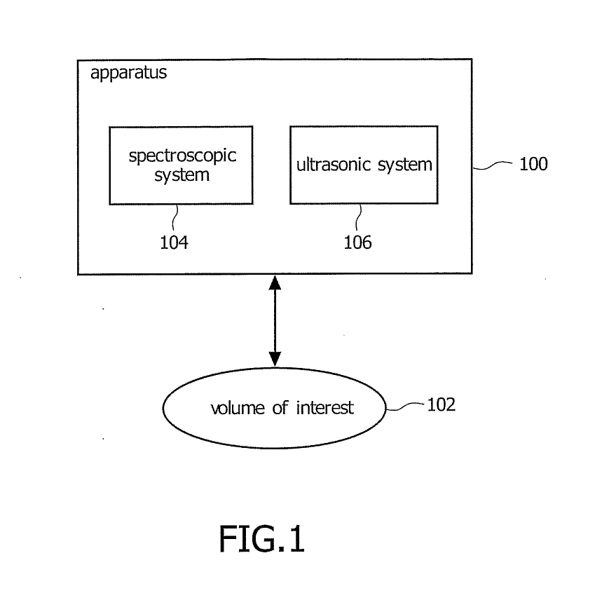

FIG. 1 shows a block diagram of the inventive apparatus,

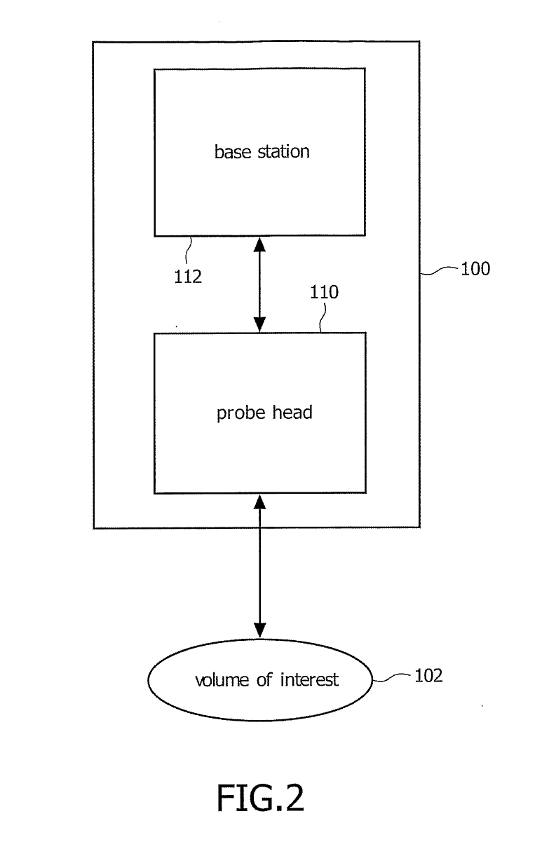

FIG. 2 shows a block diagram of the inventive apparatus having a base station and a probe head,

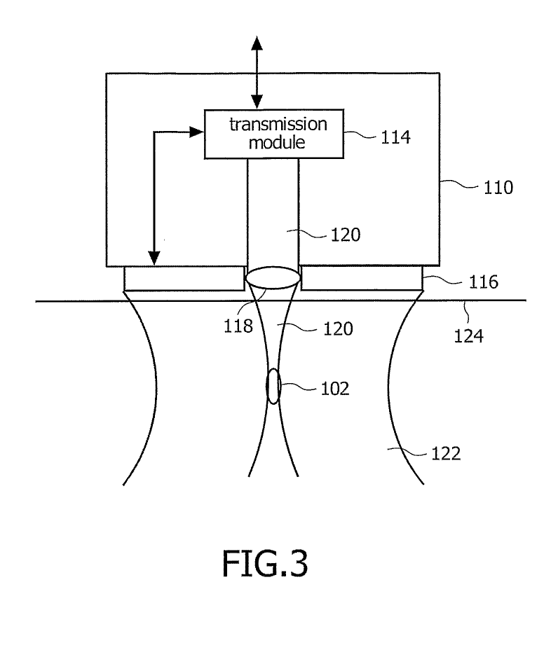

FIG. 3 shows a block diagram of the probe head attached to the surface of a skin,

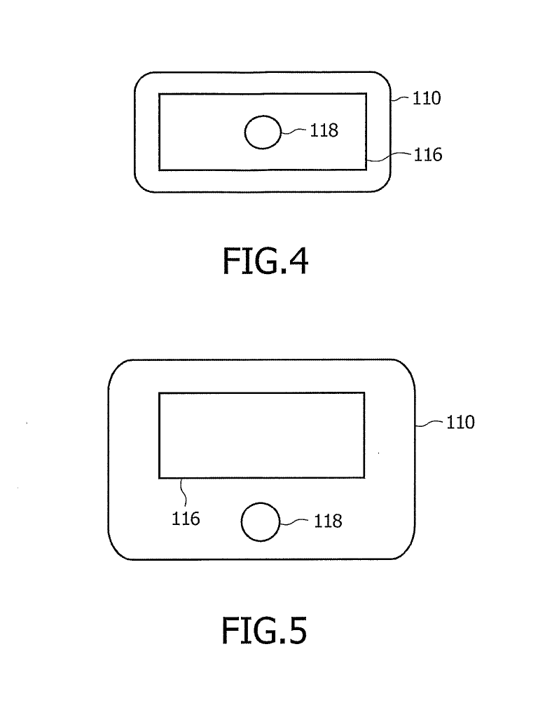

FIG. 4 is illustrative of a bottom view of the probe head,

FIG. 5 is illustrative of a bottom view of an alternative embodiment of the probe head,

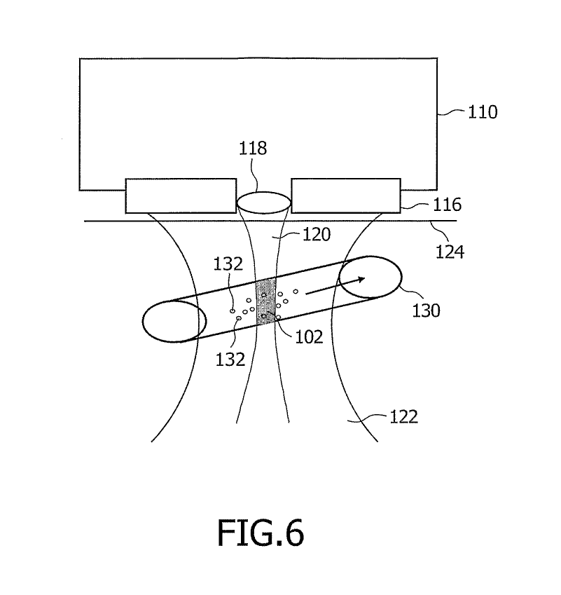

FIG. 6 shows a schematic illustration of spectroscopic and ultrasonic data acquisition making use of metal nano-particles injected into a blood vessel,

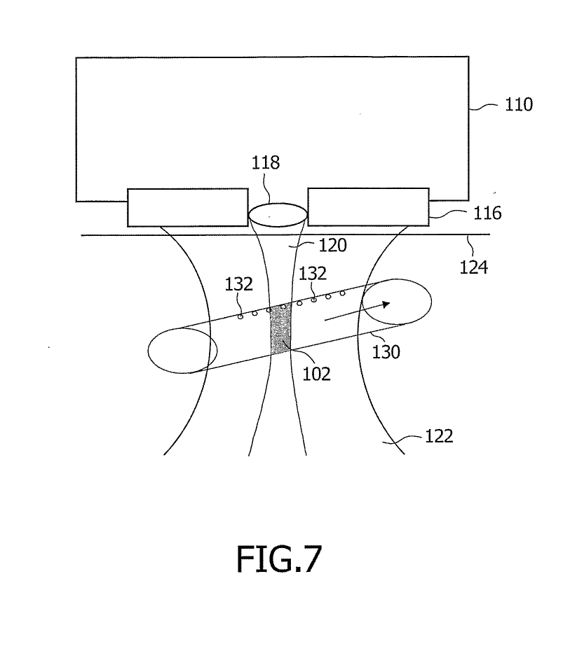

FIG. 7 shows a schematic illustration of spectroscopic and ultrasonic data acquisition making use of targeted nano-particles adsorbing to a wall of the blood vessel,

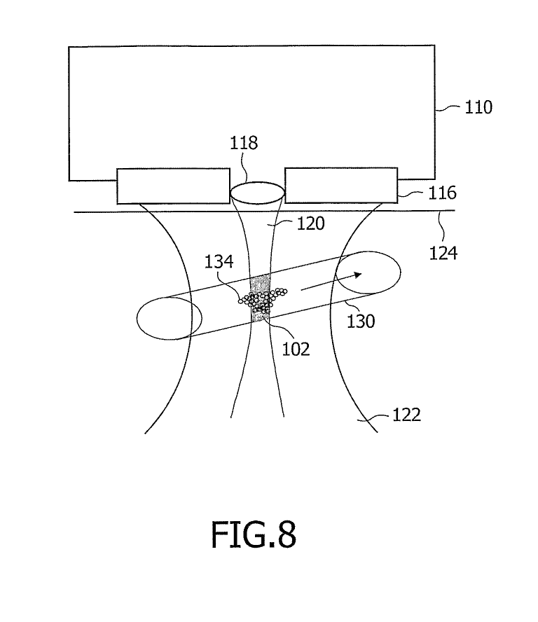

FIG. 8 shows a schematic illustration of spectroscopic and ultrasonic data acquisition making use of clustered nano-particles,

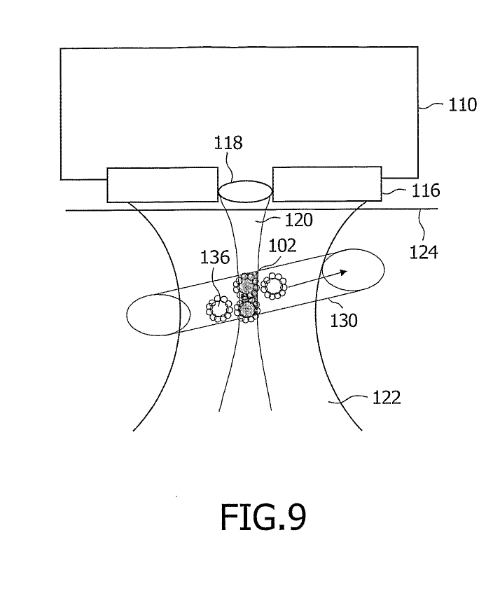

FIG. 9 shows a schematic illustration of spectroscopic and ultrasonic data acquisition making use of targeted nano-particles adhering to the shell of microbubbles,

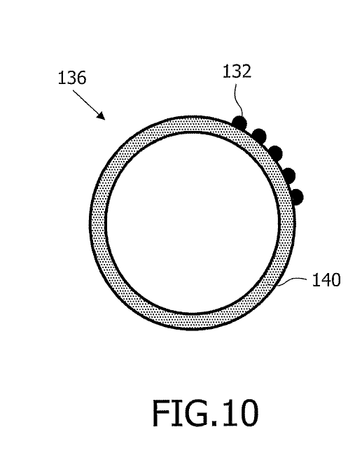

FIG. 10 schematically illustrates adhesion of metal nano-particles at the shell of a microbubble.

FIG. 1 illustrates a block diagram of an apparatus 100 comprising a spectroscopic 104 and an ultrasonic system 106. The apparatus 100 is adapted for investigation of a volume of interest 102. The ultrasonic system 106 of the apparatus 100 is adapted to acquire ultrasonic echo signals from the volume of interest in order to generate a visual illustration of the volume of interest 102. The spectroscopic system 104 in turn is adapted to spectroscopically analyze a biological structure that is located within the volume of interest 102. The spectroscopic system 104 is preferably adapted but not restricted to e.g. Raman spectroscopy. Other optical spectroscopic techniques can also be applied. These include various methods based on Raman scattering including non-linear Raman spectroscopy, such as stimulated Raman spectroscopy and coherent anti-Stokes Raman spectroscopy, infrared spectroscopy, in particular infrared absorption spectroscopy, Fourier transform infrared spectroscopy and near infrared diffusive reflection spectroscopy. Moreover, other scattering spectroscopy techniques, such as fluorescence spectroscopy, multi-photon fluorescence spectroscopy and reflectance spectroscopy and various other spectroscopic techniques such as photo-acoustic spectroscopy, polarimetry and pump probe spectroscopy can be applied. Preferred spectroscopic techniques for application to the present invention are Raman spectroscopy and fluorescence spectroscopy.

By means of the spectroscopic system 104 at least one property of the biological structure that is located within the volume of interest 102 can be determined. Preferably, the spectroscopic system allows for determining the relative weights of the molecular composition of the biological structure. When for example the volume of interest is within a cardiovascular system of a patient, the spectroscopic system is preferably adapted to determine the relative weights of e.g. cholesterol, glucose, calcium salts, blood oxygen, etc. In this way the apparatus 100 can be effectively used for detection and visualization of blood flow in combination with a composition analysis of the blood. Acquisition of both spectroscopic as well as ultrasonic signals can be performed in vivo and in a non-invasive way.

FIG. 2 illustrates a block diagram of the apparatus 100 having a probe head 110 and a base station 112. Again, the apparatus 100 is adapted to acquire and to process spectroscopic data and ultrasonic data obtained from a volume of interest 102. In comparison to FIG. 1 the base station 112 as well as the probe head 110 provide the entire functionality of the spectroscopic system 104 and the ultrasonic system 106. Preferably, the spectroscopic system 104 is divided into the base station 112 and the probe head 110 and the ultrasonic system 106 is divided accordingly. Since both the spectroscopic system 104 as well as the ultrasonic system 106 comprise data acquisition means as well as data processing means, the probe head 110 is particularly adapted for data acquisition purpose whereas the base station 112 provides appropriate means for processing of spectroscopic data and for processing of ultrasonic data. In this way the probe head 110 of the apparatus 100 can be designed in a compact way.

An extreme compact design of the probe head 110 even allows to design the entire probe head 110 as a catheter or endoscope for intravascular acquisition of data. Therefore, the probe head 110 has an ultrasonic transducer for acquisition of ultrasonic echo data and an objective lens for directing an excitation beam into the volume of interest 102 and for collecting inelastically scattered return radiation from the volume of interest 102.

The probe head 110 and the base station 112 are preferably connected by some data transmission means, such as an optical fiber and/or electrical conducting elements.

FIG. 3 schematically illustrates a cross sectional view of the probe head 110 that is attached to the surface of the skin 124 of a patient. At its bottom side facing the surface of the skin 124 the probe head 110 has an ultrasonic detection element 116 and an objective lens 118. The ultrasonic detection element 116 as well as the objective lens 118 may be designed as an integral part of the housing of the probe head 110. The probe head 110 has a transmission module 114 that is adapted to transmit the acquired ultrasonic and spectroscopic signals to the base station 112. The ultrasonic detection element 116 serves as an ultrasonic sensor, hence it is at least adapted to detect ultrasonic echo signals and may be implemented as a piezoelectric based element. Additionally, the ultrasonic detection element might be further adapted to generate and to transmit ultrasonic signals to the volume of interest. The ultrasonic sensor may therefore be implemented as an ultrasonic transducer.

The objective lens 118 is adapted to collimate an excitation beam into the volume of interest 102 and to collect inelastically scattered return radiation 120 from the volume of interest 102. The ultrasonic detection element 116, preferably implemented as ultrasonic transducer, is adapted to acquire ultrasonic echo signals from an ultrasonic acquisition area 122. The volume of interest 102 is entirely located within the ultrasonic acquisition area 122. Therefore, ultrasonic images as well as spectroscopic analysis can be simultaneously obtained from the volume of interest 102.

FIG. 4 schematically illustrates a possible embodiment of the bottom side of the probe head 110. Here, the bottom side of the probe head 110 facing the surface of the skin of a patient has a somewhat rectangular shape and has a rectangular shaped ultrasonic detection element 116 as well as a circular shaped objective lens 118. In this particular embodiment the objective lens 118 is arranged within a cut out of the ultrasonic detection element 116. In alternative embodiments, the ultrasonic detection element 116 and the objective lens 118 can also be arranged adjacent to each other. The design of the probe head 110 must only guarantee that the objective lens 118 and the ultrasonic detection element 116 do not obstruct mutually.

FIG. 5 schematically illustrates an alternative embodiment of the probe head 110, wherein the ultrasonic detection element 116 is adjacently arranged with respect to the objective lens 118. Here, the objective lens 118 is not an integrated part of the ultrasonic detection element but is positioned elsewhere at the bottom face of the probe head. In this particular embodiment, the return radiation for the spectroscopic analysis emanating from the volume of interest as well as corresponding ultrasonic echo signals must be detectable by means of the objective lens 118 and the ultrasonic detection element 116. Hence, the detection volumes for both the spectroscopic system and the ultrasonic system have to substantially overlap with the volume of interest.

FIG. 6 shows a schematic illustration of spectroscopic and ultrasonic data acquisition making use of a contrast agent 132 that has been injected into the cardiovascular system of a patient and therefore flowing within a blood vessel 130. The schematic illustration of FIG. 6 refers to an embodiment of the probe head 110 as illustrated in FIG. 3. The probe head 110 has a ultrasonic detection element 116 and an objective lens 118 for collecting return radiation 120 from the volume of interest 102. Here, the volume of interest 102 is entirely located within a blood vessel 130 underneath the surface of a skin 124 of a patient. The schematic illustration of FIG. 6 is indicative for an in vivo non-invasive blood analysis in combination with a visualization of blood flow.

The contrast agents 132 comprise solid metal nano-particles that are floating with the blood current as indicated by the arrow. The solid metal nano-particles on the one hand serve as contrast agent for the ultrasonic imaging and on the other hand provide surface enhanced Raman spectroscopy (SERS). Hence, by injection of these nano-particles both ultrasonic reflection as well as sensitivity of spectroscopic analysis is appreciably enhanced. Since the metal nano-particles may even penetrate through vessel walls or specific tissue of the patient, not only a stream of blood but also tissue in the vicinity of the blood stream can be sufficiently analyzed.

Typically, the metal particles comprise a metal, which has an acoustic impedance above 35.10⁵ g/cm²s, particularly above 50.10⁵ g/cm²s, or a mixture of one or more metals. More particularly, the metal particles are a noble metal, e.g. gold, silver, platinum. Preferably, the metal nano-particles are made of gold and have a diameter between 1 nanometer and 100 nanometers, particularly between 1 nanometer and 50 nanometers. In this way, the metal nano-particles are effectively biocompatible in a sense that they are non-toxic. Furthermore, it is known that by increasing the particle size of the metal nano-particles their ultrasonic reflection properties enhance. Additionally, applying a high ultrasound frequency results in an increased reflection enhancement. Making efficient use of the contrast agent for both SERS and ultrasound imaging, preferably a relatively high ultrasound frequency, such as e.g. up to 400 MHz, is applicable.

FIG. 7 shows a similar schematic illustration of spectroscopic and ultrasonic data acquisition as already provided by FIG. 6. Here, the contrast agents 132 are particularly designed as targeted nano-particles. This means that the contrast agents comprise a bio-target agent such as cell, tissue, microorganism, e.g. parasite, or bio-molecule, e.g. protein, DNA or RNA specific target agents of which antibodies or fragments thereof are only one example. Generally, targeted contrast agents are designed for adhesion to designated biological structures. For example they are designed to adhere to the blood vessel wall, plaque or other designated tissue within the volume of interest. FIG. 7 schematically illustrates targeted nano-particles being adsorbed by the wall of the blood vessel 130. On the one hand this provides appreciable reflection enhancement near the vessel wall for ultrasonic echo signals and on the other hand this provides SERS signals from tissue, deposited plaque or blood near the vessel wall. This is particular advantageous to selectively investigate the morphology as well as the composition of tissue of the vessel wall. Moreover, by means of this type of targeted nano-particles rupture prone atherosclerotic plaque s in coronary arteries can be precisely detected in a non-invasive way.

FIG. 8 shows a schematic illustration of spectroscopic and ultrasonic data acquisition making use of a cluster of contrast agents 134. In this case, a cluster of metal nano-particles are floating with the stream of blood within the blood vessel 130. Again a cluster of nano-particles serves as contrast agent and provides enhancement of ultrasound echo signals as well as enhancement of Raman spectroscopic signals. Especially when making use of targeted nano-particles that are adapted to adhere to specific tissue, also an entire cluster formed of targeted nano-particles may effectively become a targeted cluster. In this way ultrasound echo signals and Raman spectroscopic signals are effectively enhanced for specific tissue that is of particular interest. Hence, tissue specific contrast and signal enhancement can principally be realized.

FIG. 9 shows a schematic illustration of spectroscopic and ultrasonic data acquisition wherein micro sized gas bubbles 136 as well as metal nano-particles are injected into the stream of blood. Preferably, the gas bubbles 136 are encapsulated by the metal nano-particles and therefore act as conventional contrast agents for ultrasound imaging. The metal nano-particles in turn provide a surface enhanced spectroscopic effect and therefore the functionality of the gas bubbles 136 becomes twofold. First, the gas bubbles themselves serve as a conventional contrast agents for ultrasound imaging. Second, the nano-sized metal particles adhered at the shell of the gas bubbles allow for a sufficient surface enhancing spectroscopic effect. In this way reflection enhancement of ultrasonic echo signals is mainly provided by the gas bubbles with a diameter in the range of micrometers and surface enhanced Raman spectroscopy is supported by the metal nano-particles adsorbed to the shell of the gas bubbles. The diameter of these gas bubbles is typically in the range of a few micrometers, preferably between 1 and 8 micrometers. Additionally, the bubbles have a protective membrane shell to increase their lifetime.

Alternatively, gas bubbles and metal particles can also be injected separately into the stream of blood of a patient. When injected into the blood stream both metal nano particles as well as gas bubbles then selectively serve as agents for enhancing spectroscopic and ultrasonic signals, respectively. In this way adhesion of the nano-particles to the membrane of the gas bubbles does not necessarily have to be prepared.

FIG. 10 illustrates a cross section of a gas bubble 136 having a shell 140 that is adapted for adhesion of metal nano-particles 132. In this way the relatively large shell 140 of the gas bubble 136 mainly serves to enhance reflection efficiency of the contrast agent for ultrasonic imaging whereas the metal nano-particles 132 featuring a size in the range of nanometers provide efficient surface enhancement of Raman spectroscopic signals.

Hence, by making use of targeted metal nano-particles 132 adhering to the shell 140 of a gas bubble, the entire resulting gas bubble shell with adhered metal nano-particles may effectively become a targeted cluster 136 that is adapted to adhere to target specific tissue. Also in this way, tissue specific contrast and signal enhancement referring to ultrasound echo signals and Raman spectroscopic signals can be realized.

### LIST OF REFERENCE NUMERALS

100 apparatus
102 volume of interest
104 spectroscopic system
106 ultrasonic system
110 probe head
112 base station
114 transmission module
116 ultrasonic detection element
118 objective lens
120 return radiation
122 ultrasonic acquisition area
124 skin surface
130 blood vessel
132 contrast agent
134 cluster of contrast agents
136 gas bubble
140 shell

1. An apparatus (100) comprising:
an ultrasonic imaging system (106) for imaging of a volume of interest (102),
a spectroscopic system (104) for determining a property of a biological structure being located within the volume of interest.

2. The apparatus according to claim 1, further comprising a flexible probe head (110) having:
an objective lens (118) for directing an excitation beam into the volume of interest (102) and for collecting return radiation (120) from the volume of interest,
an ultrasonic detection element (116) for acquisition of ultrasonic echo signals from the volume of interest during a period of time.

3. The apparatus according to claim 1, further comprising a base station (112) having:
a spectroscopic analysis unit being adapted to perform a spectroscopic analysis of the return radiation,
an ultrasonic analysis unit being adapted to generate an image of the volume of interest on the basis of the ultrasonic echo signals.

4. The apparatus according to claim 1, further being adapted to perform spectroscopic analysis of the biological structure by means of the spectroscopic system (104) and to simultaneously generate an image of the volume of interest by means of the ultrasonic imaging system (106).

5. The apparatus according to claim 2, wherein the flexible probe head (110) being implemented as a hand-held device being adapted to be attached to the surface of a skin (124) of a patient.

6. The apparatus according to claim 2, wherein the flexible probe (110) head being implemented as a catheter being adapted to be inserted into a cardiovascular system (130) of a patient.

7. The apparatus according to claim 1, wherein the ultrasonic imaging system (106) being adapted to acquire and to process ultrasonic echo signals from solid metal nano-particles (132) being injected into the volume of interest as a contrast agent.

8. The apparatus according to claim 7, wherein the spectroscopic system (104) being adapted to acquire and to process return radiation (120) that is due to a surface enhanced Raman scattering process induced by the solid metal nano-particles (132).

9. A probe head (110) of a diagnostic apparatus (100) for imaging a volume of interest (102) and for determining a property of a biological structure being located within the volume of interest, the probe head comprising:
an objective lens (118) for directing an excitation beam into the volume of interest and for collecting return radiation from the volume of interest,
an ultrasonic detection element (116) for acquisition of ultrasonic echo signals from the volume of interest during a period of time.

10. The probe head according to claim 9, wherein the objective lens (118) being an integral part of the ultrasonic detection element (116).

11. A base station (112) of a diagnostic apparatus (100) for imaging a volume of interest (102) and for determining a property of a biological structure being located within the volume of interest, the base station comprising:
a spectroscopic analysis unit being adapted to perform spectroscopic analysis of return radiation (120) returning from the volume of interest,
an ultrasonic analysis unit being adapted to generate an image of the volume of interest on the basis of ultrasonic echo signals from the volume of interest.

12. A method of imaging a volume of interest (102) and for determining a property of a biological structure being located within the volume of interest, the method comprising the steps of:
acquiring of ultrasonic echo signals from the volume of interest during a period of time for generating an ultrasonic image of the volume of interest,
directing an excitation beam into the volume of interest and collecting return radiation (120) from the volume of interest,
performing spectroscopic analysis of the collected return radiation in order to determine a property of a biological structure being located within the volume of interest.

13. The method according to claim 12, further comprising injecting a contrast agent of solid metal nano-particles (132) into the volume of interest, the contrast agent being adapted to enhance the contrast of ultrasonic echo signals and to simultaneously enhance the intensity of the return radiation (120).

14. The method according to claim 13, wherein the metal nano-particles (132) comprising a bio-targeted agent attached to the surface of the metal nano-particle.

15. The method according to claim 12, further comprising injecting of micro sized gas bubbles (136) with metal nano-particles (132) as a contrast agent into the volume of interest, the gas bubbles being adapted to enhance the reflection efficiency of ultrasonic echo signals and the metal nano-particles are adapted to enhance the intensity of the return radiation (120).
